(19) United States
(12) Patent Application Publication
(10) Pub. No.: US 2004/0236435 A1
(43) Pub. Date: Nov. 25, 2004

(54) Prosthetic foot

(75) Inventor: Sen-Jung Chen, Taipei City (TW)

Correspondence Address: LADAS & PARRY, 5670 WILSHIRE BOULEVARD, SUITE 2100, LOS ANGELES, CA, 90036-5679, US

(73) Assignee: TEH LIN PROSTHETIC & ORTHOPAEDIC INC.

(21) Appl. No.: 10/442,310

(22) Filed: May 19, 2003

(51) Int. Cl. A61F 2/66; A61F 2/74; A61F 2/62

(52) U.S. Cl. 623/49; 623/38; 623/56

(57) ABSTRACT

A prosthetic foot includes a foot body having heel and toe parts, an ankle member mounted pivotally on the heel part and having two opposite ends and an intermediate part between the opposite ends, an ankle shaft extending through the intermediate part of the ankle member, and two biasing members mounted within the heel part below the ankle member and respectively adjacent to the opposite ends so as to support the ankle member. A hydraulic bumper unit may be provided to support the biasing members.

### BACKGROUND OF THE INVENTION

1. Field of the Invention

The invention relates to a prosthetic foot, more particularly to a prosthetic foot having an energy restoring compression spring and a universal joint in an ankle member.

2. Description of the Related Art

Referring to FIG. 1, a conventional prosthetic foot includes a foot body 1, a retaining unit 2, an ankle member 3, a horizontal shaft 4 interconnecting pivotally the retaining unit 2 and the ankle member 3, and two shock-absorbing pieces 5 disposed between the ankle member 3 and the foot body 1. The retaining unit 2 has a lower threaded shank portion 201, an upper ring portion 202 connected fixedly to a top portion of the shank portion 201, a ball bearing 203 disposed in the ring portion 202, and two supporting pads 204 disposed on each side of the ring portion 202 so as to center the retaining unit 2 relative to the ankle member 3. The ball bearing 203 includes a ring seat 2031 mounted fittingly in the ring portion 202, and a single ball 2032 disposed rotatably in the ring seat 2031. The shaft 4 extends through the ankle member 3, the ball 2032, and the supporting pads 204. The shock-absorbing pieces 5 are made of hard rubber material, and provide stability and shock-absorbing effects to the ankle member 3 relative to the foot body 1.

Although the conventional prosthetic foot described above is provided with a universal joint, since the shaft 4 that interconnects the retaining unit 2 and the ankle member 3 is in a horizontal direction, the retaining unit 2 has to be centered to provide good balance for the user. However, the centering of the retaining unit 2 requires additional component parts. Furthermore, the foot body 1 has to provide a receiving space that is relatively large so as to receive the lower part of the retaining unit 2 which covers the horizontal shaft 4 and the additional components.

### SUMMARY OF THE INVENTION

Therefore, the main object of the present invention is to provide a prosthetic foot that is capable of overcoming the aforementioned drawbacks of the prior art.

Another object of the present invention is to provide a prosthetic foot with a hydraulic bumper unit to improve shock-absorbing characteristics.

According to the present invention, a prosthetic foot comprises a foot body having a heel part and a toe part, an ankle member mounted pivotally on the heel part and having two opposite ends and an intermediate part between the opposite ends, an ankle shaft extending through the intermediate part of the ankle member, and two biasing members mounted within the heel part below the ankle member and respectively adjacent to the opposite ends so as to support the ankle member.

### BRIEF DESCRIPTION OF THE DRAWINGS

Other features and advantages of the present invention will become apparent in the following detailed description of the preferred embodiments with reference to the accompanying drawings, of which:

FIG. 1 is an exploded perspective view of a conventional prosthetic foot;

FIG. 2 is an exploded perspective view of the first preferred embodiment of a prosthetic foot according to the present invention;

FIG. 3 is a sectional view of the first preferred embodiment in an assembled state;

FIG. 4 is the same view as FIG. 3, but with the heel part of the foot body being lifted away from the ground surface;

FIG. 5 is an exploded perspective view of the second preferred embodiment of a prosthetic foot according to the present invention;

FIG. 6 is a sectional view of the second preferred embodiment in an assembled state; and

FIG. 7 is a sectional view taken along line 7-7 of FIG. 6, illustrating a hydraulic bumper unit of the second preferred embodiment.

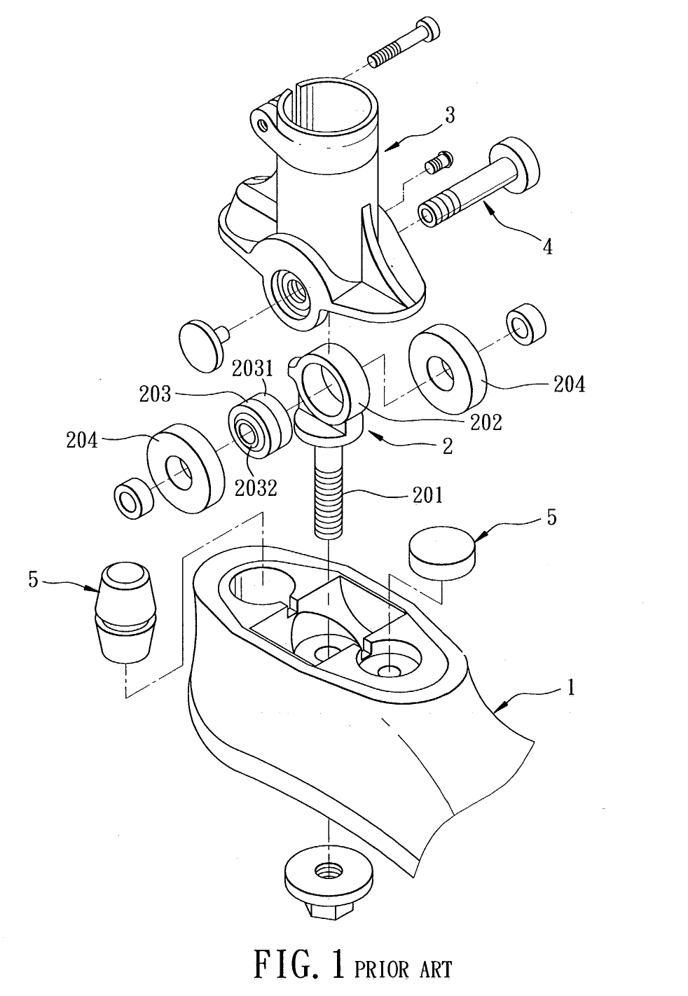

### DETAILED DESCRIPTION OF THE PREFERRED EMBODIMENTS

Before the present invention is described in greater detail, it should be noted that like elements are denoted by the same reference numerals throughout the disclosure.

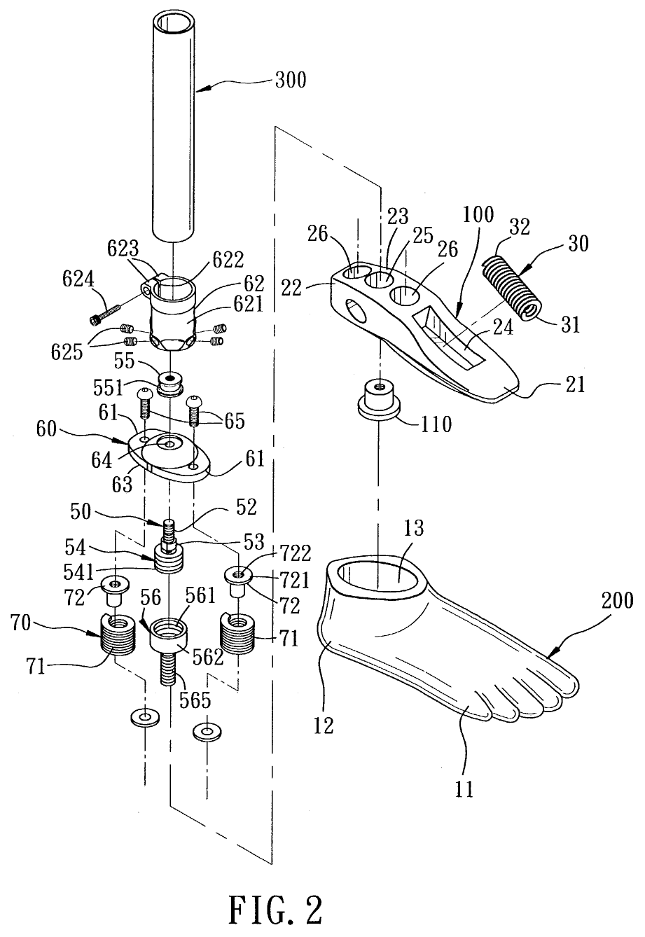

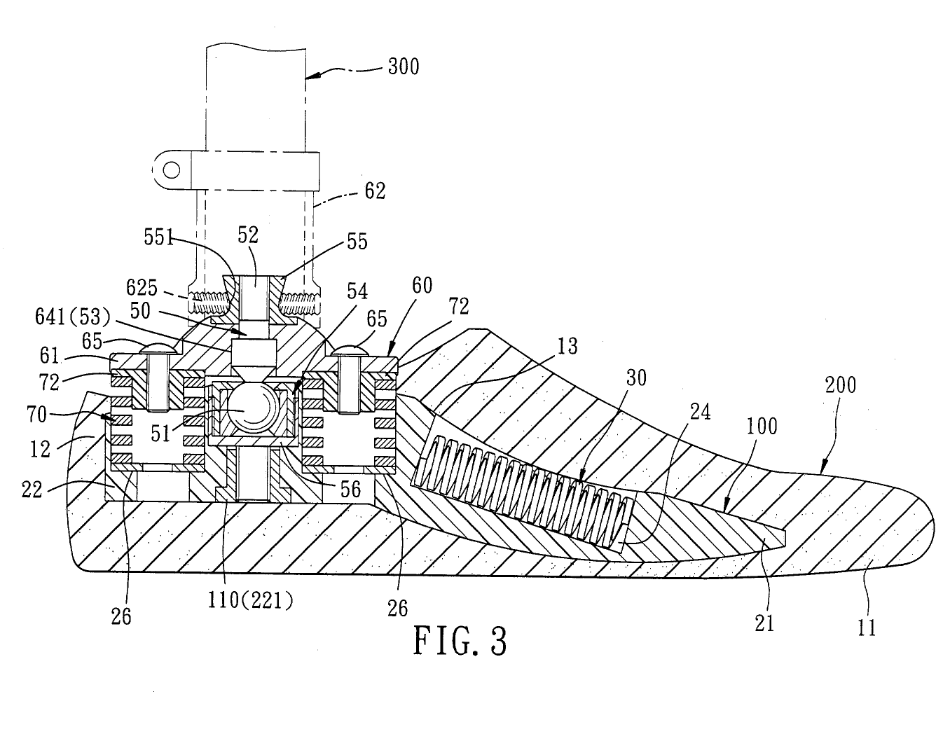

Referring to FIGS. 2 and 3, the first preferred embodiment of a prosthetic foot according to the present invention is adapted to be connected to a leg member 300, and is shown to comprise a foot body 100, an ankle shaft 50, an ankle member 60, a shin 62, two spring members 70, and a shell 200 around the foot body 100.

The foot body 100 is made of a plastic material, and includes a toe part 21, a heel part 22 opposite to the toe part 21, an upper surface 23 that extends from the toe part 21 to the heel part 22, a longitudinal cavity 24 between the toe part and the heel part 21, 22 and extending inwardly from the upper surface 23, and a spring mounted in the longitudinal cavity 24 and having a first end 31 extending toward the toe part 21 and a second end 32 extending toward the heel part 22. The heel part 22 has a joint hole 25, and two spring holes 26 on two sides of the joint hole 25. The cavity 24 has opposite front and rear cavity walls 241, 242. The spring is mounted in the longitudinal cavity 24, and in this embodiment, is a helical compression spring 30. The first and second ends 31, 32 of the spring 30 abut respectively against the front and rear cavity walls 241, 242 of the longitudinal cavity 24.

The ankle shaft 50 has a bottom end extending into the joint hole 25 and formed with a ball 51 (see FIG. 3), a screw portion 52 opposite to the ball 51, a positioning part 53 between the screw portion 52 and the ball 51, a socket 54 receiving the ball 51 to form a ball-and-socket joint, a nut 55 attached to the screw portion 52, and a retaining unit 56. The positioning part 53 has a multi-sided cross section. The nut 55 has an outer periphery formed with an annular groove 551. The retaining unit 56 is fixed to the heel part 22 within the joint hole 25 for retaining the socket 54 within the joint hole 25, and includes a receiving space 561 and a retaining wall 562 confining the receiving space 561. The socket 54 has a socket wall with an outer wall face 541 engaging threadedly the retaining wall 562. The retaining unit 56 is screwed to the heel part 22 by means of a screw rod 565 which is formed integrally with the retaining wall 562, and extends downward to threadedly engage a female screw member 110 received in a bottom hole 221 (see FIG. 3) formed at the bottom of the heel part 22.

The ankle member 60 is mounted pivotally on the heel part 22, and has two opposite ends 61, an intermediate part 63 between the opposite ends 61 and having a dome-shaped top surface, and a through hole 64 formed in the intermediate part 63. The ankle shaft 50 extends through the through hole 64 in such a manner that the screw portion 52 and the ball 51 of the ankle shaft 50 are located respectively above and below the intermediate part 63 of the ankle member 60. The nut 55 engages the screw portion 52 of the ankle shaft 50 to lock the ankle member 60 relative to the ankle shaft 50. The through hole 64 has a part 641 (see FIG. 3) for receiving the positioning part 53, and has a cross-section substantially corresponding to the multi-sided cross section of the positioning part 53. The part 641 of the through hole 64 receives the positioning part 53 so as to prevent relative rotational movement between the ankle shaft 50 and the ankle member 60.

The prosthetic foot of the present invention further includes a shin 62. The shin 62 includes a tubular wall 621 extending around the screw portion 52 and the nut 55, and a plurality of clamping screws 625 extending radially and threadedly through the tubular wall 621 and having ends engaging the annular groove 551 in the nut 55. The tubular wall 621 has a split 622, two clamping lugs 623 integrally formed with the tubular wall 621 adjacent to two sides of the split 622, and a clamping screw 624 extending threadedly through the clamping lugs 623 so that the tubular wall 621 can be clamped tightly against the leg member 300.

The biasing members 70 are mounted within the heel part 22 of the foot body 100 below the ankle member 60 and are respectively adjacent to the opposite ends 61 of the ankle member 60 so as to support the latter. Each of the biasing members 70 is a spring coil 71. Each of the spring coils 71 is received in a corresponding one of the spring holes 26 in the heel part 22, and incorporates a hollow screw seat 72 snugly fitted into the corresponding spring coil 71. Each screw seat 72 has a top flange 721 seated on top of a respective one of the spring coils 71, and a female screw 722 formed inside the screw seat 72. The ankle member 60 is screwed to the screw seats 72 of the spring coils 71 by means of a pair of screws 65 that extend respectively through through holes in the opposite ends 61 of the ankle member 60 and that engage respectively the female screws 722 of the screw seats 72.

The shell 200 is made of a flexible foam material, and includes a front end portion 11, a rear end portion 12, and a receiving space 13 extending from a top surface of the rear end portion 12 to the front end portion 11. The foot body 100 is inserted into the receiving space 13 in the shell 200, and has the toe part 21 thereof extending toward the front end portion 11 of the shell 200.

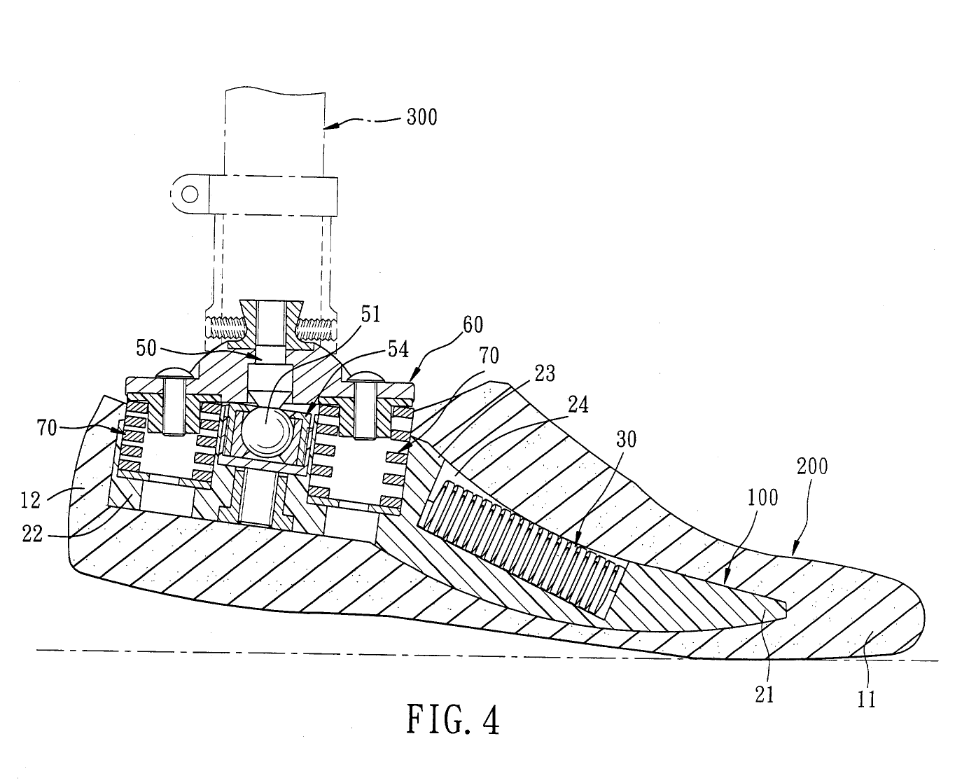

When the user, after wearing the prosthetic foot of the present invention, strides during walking on the ground surface in a manner in which the front end portion 11 of the shell 200 presses against the ground surface and the rear end portion 12 of the shell 200 is raised, the heel part 22 of the foot body 100 deflects upward relative to the toe part 21, thus causing the upper surface 23 to deform and contract the cavity 24. The biasing members 70 react in a manner shown in FIG. 4. The helical spring 30, at this time, is subjected to a compression force to store energy. Upon continued walking, the prosthetic foot is lifted, and the ground-pressing force is relieved. In this situation, the helical spring 30 biases the foot body 100 to restore the upper surface 23 from a deflected state to a normal state. At the same time, the leg member 300 moves pivotally along with the ankle member 60 and the ankle shaft 50 relative to the foot body 100 for restoring to an original position shown in FIG. 3.

The advantages of the first preferred embodiment of the prosthetic foot of the present invention can be summarized as follows:

1. Due to the configuration of the retaining unit 56 that engages threadedly the socket 54, and the vertical ankle shaft 50 that is integral with a ball-and-socket joint, the prosthetic foot of the present invention is simple and easy to assemble and repair.

2. The vertical ankle shaft 50 can be centered with respect to the shin 62 using fewer components as compared to that of the conventional prosthetic foot.

3. Resiliency of the foot body 100 can be adjusted by selecting a helical spring 30 with a suitable number of turns. The number of turns of the helical spring 30 determines the resiliency of the foot body 100 to match the weight of the wearer.

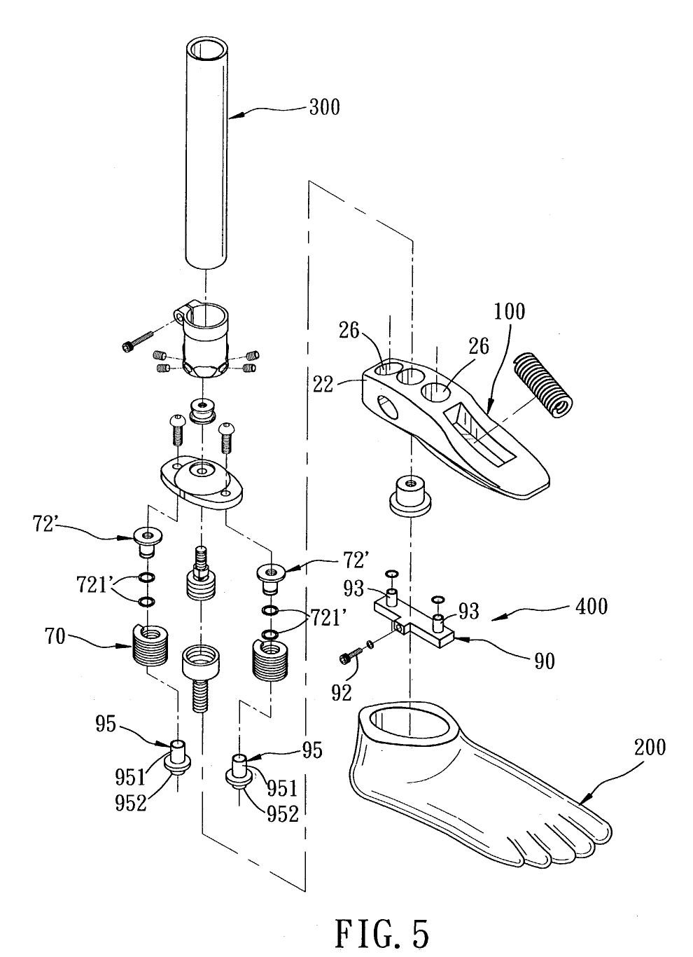

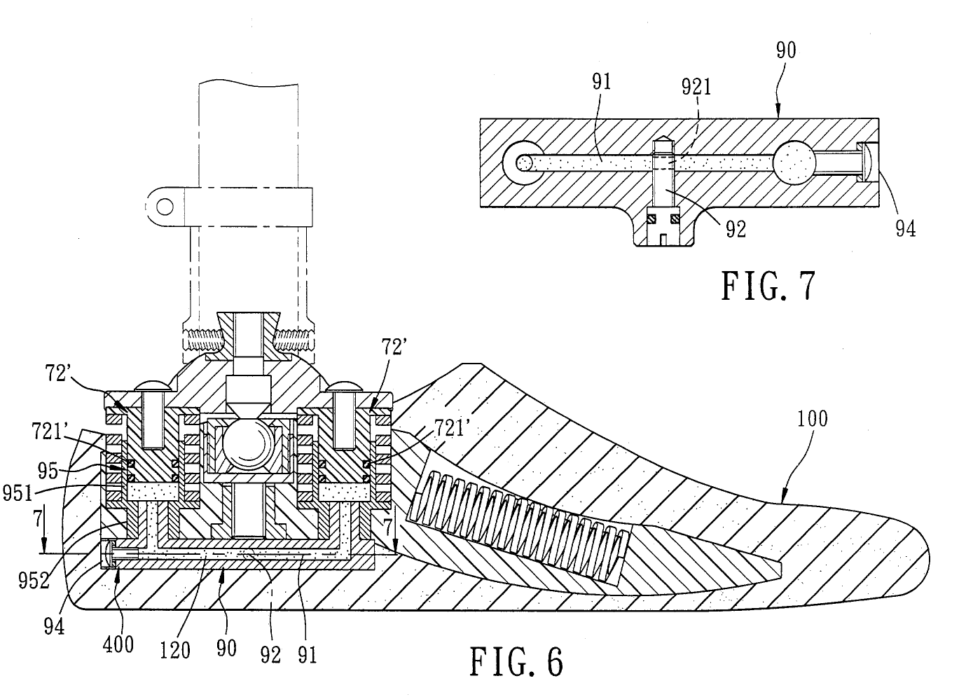

Referring to FIGS. 5 to 7, the second preferred embodiment of the prosthetic foot according to the present invention is shown to be substantially similar to the first preferred embodiment. However, in this embodiment, the prosthetic foot further comprises a hydraulic bumper unit 400, which includes a hydraulic seat 90 mounted on the foot body 100 below the biasing members 70, and a hydraulic fluid 120. The hydraulic seat 90 is disposed at a bottom side of the foot body 100, and includes two spaced-apart upright fluid receiving parts extending upward from the hydraulic seat 90, a fluid passage 91 formed within the hydraulic seat 90 between and in fluid communication with the fluid receiving parts, a valve 92 disposed across the passage 91, and a fluid injection hole 94 in fluid communication with the passage 91. The fluid receiving parts are respectively connected to the biasing members 70 so as to provide support to the biasing members 70. Each of the fluid receiving parts includes a tubular post 93 and a tubular sleeve 95. The tubular posts 93 extend integrally from a top surface of the hydraulic seat 90, and extend respectively into the spring holes 26 in the heel part 22 of the foot body 100. Each of the tubular sleeves 95 has an upper portion 951 that receives sealingly a respective one of the screw seats 72′, and a lower portion 952 that is sleeved sealingly around a respective one of the tubular posts 93. The fluid 120 is received within the tubular post 93 and the upper portion 951 of the tubular sleeve 95 to exert a fluid pressure on the screw seat 72′ so that the screw seats 72′ can act like pistons.

The valve 92 has a through hole 921, and can be operated manually to turn between a fully opened position, in which the through hole 921 is aligned with the fluid passage 91, and a fully closed position, in which the through hole 921 is transverse to the fluid passage 91.

The amount of the fluid 120 flowing through the through hole 921 in the valve 92 can be adjusted by rotating the valve 92. As such, the fluid 120 that flows into the tubular posts 93 from the fluid passage 91 can be controlled so as to suit the weight of the user. When the valve 92 is rotated to the fully opened position, the fluid 120 flowing through the through hole 921 is at a maximum value, and the hydraulic bumper unit 400 provides a maximum softness which is suitable for a light-weight user.

Each screw seat 72′ in this embodiment is snugly fitted into a corresponding spring coil 71, and extends into the upper portion 951 of the corresponding sleeve 95. A pair of seal rings 721′ are disposed between each screw seat 72′ and the upper portion 951 of the corresponding tubular sleeve 95 so that the fluid 120 is prevented from flowing out of the upper portions 951 of the tubular sleeves 95.

The advantages of the first preferred embodiment can be similarly attained by the second preferred embodiment of the present invention. Additionally, the prosthetic foot of the present invention can provide comfort to the user during use due to the presence of the hydraulic bumper unit 400.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

I claim:

1. A prosthetic foot comprising:
a foot body having a heel part and a toe part;
an ankle member mounted pivotally on said heel part and having two opposite ends and an intermediate part between said opposite ends;
an ankle shaft extending through said intermediate part of said ankle member; and
two biasing members mounted within said heel part below said ankle member and respectively adjacent to said opposite ends so as to support said ankle member.

2. The prosthetic foot as claimed in claim 1, wherein said foot body further has an upper surface that extends from said toe part to said heel part, a longitudinal cavity between said toe part and said heel part and extending inwardly from said upper surface, and a spring mounted in said longitudinal cavity and having a first end extending toward said toe part and a second end extending toward said heel part, wherein said foot body is deflectable to deform said upper surface and to contract said cavity, and wherein said spring is subjected to a compression force when said cavity contracts, and restores said upper surface to a normal position thereof when the compression force is relieved.

3. The prosthetic foot as claimed in claim 2, wherein said heel part has a joint hole, said ankle shaft having a bottom end extending into said joint hole and formed with a ball below said intermediate part, a socket receiving said ball to form a ball-and-socket joint, and a retaining unit fixed to said heel part within said joint hole for retaining said socket within said joint hole.

4. The prosthetic foot as claimed in claim 2, wherein said ankle shaft further includes a screw portion above said ankle member and a nut attached to said screw portion for locking said ankle member relative to said ankle shaft.

5. The prosthetic foot as claimed claim 4, wherein said ankle shaft further includes a positioning part between said screw portion and said ball, said positioning part having a multi-sided cross section, said ankle member further having a through hole for passage of said ankle shaft, said through hole having a part for receiving said positioning part and having a cross-section substantially corresponding to said multi-sided cross section, said part of said through hole receiving said positioning part so as to prevent relative rotational movement between said ankle shaft and said ankle member.

6. The prosthetic foot as claimed in claim 4, wherein said nut has an outer periphery formed with an annular groove.

7. The prosthetic foot as claimed in claim 6, further comprising a shin which includes a tubular wall extending around said screw portion and said nut, and a plurality of clamping screws extending radially and threadedly through said tubular wall and having ends engaging said annular groove.

8. The prosthetic foot as claimed in claim 1, further comprising a hydraulic bumper unit, which includes
a hydraulic seat mounted on said foot body below said biasing members and including two spaced-apart upright fluid receiving parts extending upward from said hydraulic seat, and a fluid passage formed within said hydraulic seat between and in fluid communication with said fluid receiving parts, said fluid receiving parts being respectively connected to said biasing members; and
a hydraulic fluid received inside said passage and said fluid receiving parts.

9. The prosthetic foot as claimed in claim 8, wherein each of said biasing members is a spring coil which incorporates a hollow screw seat fitted into said spring coil, said screw seat having a top flange seated on top of said spring coil and a female screw formed inside said screw seat, said ankle member being screwed to said screw seat through said female screw.

10. The prosthetic foot as claimed in claim 9, wherein said heel part further has a joint hole that permits extension of said ankle shaft thereinto, said foot body further having two spring holes on two sides of said joint hole, said spring coil of each of said biasing members being received in a corresponding one of said spring holes.

11. The prosthetic foot as claimed in claim 10, wherein each of said fluid receiving parts includes a tubular post extending integrally from a top surface of said hydraulic seat, and a tubular sleeve having an upper portion that receives sealingly a respective one of said screw seats, and a lower portion that is sleeved sealingly around a respective one of said tubular posts, said fluid being received within said tubular post and said upper portion of said tubular sleeve to exert a fluid pressure on said screw seat.

12. The prosthetic foot as claimed in claim 11, wherein said ankle shaft has a bottom end extending into said joint hole and formed with a ball below said intermediate part, a socket receiving said ball to form a ball-and-socket joint, and a retaining unit fixed to said heel part within said joint hole for retaining said socket within said joint hole.

13. The prosthetic foot as claimed in claim 12, wherein said retaining unit includes a receiving space and a retaining wall confining said receiving space, said socket having a socket wall with an outer wall face engaging threadedly said retaining wall.

14. The prosthetic foot as claimed in claim 13, wherein said retaining unit is screwed to said heel part.

15. The prosthetic foot as claimed in claim 14, wherein said hydraulic seat is disposed at a bottom side of said foot body, said tubular posts extending respectively into said spring holes, said upper portions of said tubular sleeves extending respectively into said spring coils to sleeve on said screw seats.

16. The prosthetic foot as claimed in claim 15, further comprising a shell around said foot body.

17. The prosthetic foot as claimed in claim 8, wherein said hydraulic seat further includes a valve disposed across said fluid passage to control the amount of said hydraulic fluid flowing from said fluid passage to said upright fluid receiving parts.
